(19) United States
(12) Patent Application Publication
(10) Pub. No.: US 2021/0109074 A1
(43) Pub. Date: Apr. 15, 2021

(54) GAS MEASUREMENT INSTRUMENT ON UNMANNED VEHICLE

(71) Applicant: SeekOps Inc., Austin, TX (US)

(72) Inventors: Brendan James Smith, Lakeway, TX (US); Vlad Manzatianu, Austin, TX (US)

(21) Appl. No.: 17/069,989

(22) Filed: Oct. 14, 2020

Related U.S. Application Data
(60) Provisional application No. 62/914,697, filed on Oct. 14, 2019.

(51) Int. Cl. G01N 33/00; B25J 13/08; B25J 19/00; B62D 57/032; G05D 1/00

(52) U.S. Cl. CPC G01N 33/0062; B25J 13/087; B25J 19/0075; G05D 2201/0207; G05D 1/0022; G01N 2033/0068; B62D 57/032

(57) ABSTRACT

Systems, devices, and methods including: an unmanned vehicle; a gas sensor attached to the unmanned vehicle, where the gas sensor is configured to measure ambient gas concentrations; and a ground control system (GCS), where the GCS is configured to display a location of the unmanned vehicle and a corresponding real-time ambient gas concentration detected by the gas sensor; and where the gas sensor is attached to the unmanned vehicle such that the gas sensor does not impair movement of the unmanned vehicle.

### CROSS-REFERENCE TO RELATED APPLICATIONS

This application claims priority to and the benefit of U.S. Provisional Patent Application No. 62/914,697, filed Oct. 14, 2019, the contents of which are hereby incorporated by reference herein for all purposes.

### TECHNICAL FIELD

Embodiments relate generally to gas measurement, and more particularly to a gas measurement instruments on mobile platforms.

### BACKGROUND

Methane (CH4) is an odorless and colorless naturally occurring organic molecule, which is present in the atmosphere at average ambient levels of approximately 1.85 ppm as of 2018 and is projected to continually climb. Methane is a powerful greenhouse gas, a source of energy (i.e., methane is flammable), and an explosion hazard, and so detection of methane is of utility to scientists as well as engineers. While methane is found globally in the atmosphere, a significant amount is collected or “produced” through anthropogenic processes including exploration, extraction, and distribution of petroleum resources as a component in natural gas. Natural gas, an odorless and colorless gas, is a primary fuel used to produce electricity and heat. The main component of natural gas is typically methane, and the concentration of methane in a stream of natural gas can range from about 70% to 90%. The balance of the gas mixture in natural gas consists of longer chain hydrocarbons, including ethane, propane, and butane, typically found in diminishing mole fractions that depend on the geology of the earth from which the gas is extracted. Once extracted from the ground, natural gas is processed into a product that must comply with specifications for both transport, taxation, and end-use in burners;

specification of processed ‘downstream’ natural gas product control for the composition of the gas, so as to protect transport lines from corrosion and ensure proper operation of burners and turbines. While extraction of natural gas is one of the main sources of methane in the atmosphere, major contributors of methane also include livestock farming (i.e., enteric fermentation) and solid waste and wastewater treatment (i.e., anaerobic digestion). Anaerobic digestion and enteric fermentation gas products consist primarily of methane and lack additional hydrocarbon species.

### SUMMARY

A system embodiment may include: at least one trace gas sensor configured to measure trace gas concentrations, where the at least one trace gas sensor may be configured to generate a first data and a second data packet of two or more data packets, where the first data packet and the second data packet comprise trace gas concentration data; a control electronics in communication with the at least one trace gas sensor, where the control electronics comprises a processor configured to:

process the first data packet and the second data packet to include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected; store, the first data packet in a memory of the control electronics; and transmit the second data packet to a ground control system (GCS) via a transmitter of the control electronics; an unmanned vehicle, where the at least one trace gas sensor may be attached to the unmanned vehicle such that the at least one trace gas sensor does not impair movement of the unmanned vehicle; where the GCS may be configured to display a location of the unmanned vehicle and a corresponding real-time trace gas concentration detected by the at least one trace gas sensor.

In additional system embodiments, the unmanned vehicle may be a quadrupedal robot. In additional system embodiments, the unmanned vehicle may be a ground robot. In additional system embodiments, the at least one trace gas sensor extends beyond a body width of the unmanned vehicle. In additional system embodiments, the at least one trace gas sensor may be configured to measure trace gas concentrations of at least one of: methane, ethane, propane, butane, and natural gas.

In additional system embodiments, the at least one trace gas sensor further comprises: a cover configured to protect a sensor of the trace gas sensor, where the cover comprises one or more air holes for allowing airflow into the sensor while protecting the sensor from impacts and dust; one or more mounting attachments for connecting the at least one trace gas sensor to the unmanned vehicle; and one or more extenders configured to place the sensor distal from the one or more mounting attachments. In additional system embodiments, the one or more extenders comprise a parallel pair of rods.

In additional system embodiments, the memory of the control electronics comprises a micro SD card. In additional system embodiments, the GCS may be further configured to transmit the received second data packet to a cloud server. In additional system embodiments, the GCS may be further configured to store the second data packet in a memory of the GCS.

Additional system embodiments may include: an adapter, where the adapter provides power to the at least one trace gas sensor and the control electronics from a power supply of the unmanned vehicle. Additional system embodiments may include: an adapter, where the adapter provides data for at least one: the timestamp for when the elevated trace gas concentration was detected, the latitude and longitude when the elevated trace gas concentration was detected, the altitude when the elevated trace gas concentration was detected, the temperature when the elevated trace gas concentration was detected, a wind speed when the elevated trace gas concentration was detected, a wind direction when the elevated trace gas concentration was detected, and the pressure when the elevated trace gas concentration was detected from the unmanned vehicle.

In additional system embodiments, processing the first data packet and the second data packet includes: the timestamp for when the elevated trace gas concentration was detected, the sensor time when the elevated trace gas concentration was detected, and the trace gas concentration. In additional system embodiments, processing the first data packet and the second data packet further includes: the latitude and longitude when the elevated trace gas concentration was detected and the altitude when the elevated trace gas concentration was detected. In additional system embodiments, processing the first data packet and the second data packet further includes: the temperature when the elevated trace gas concentration was detected and the pressure when the elevated trace gas concentration was detected.

A method embodiment may include: detecting, by at least one trace gas sensor, an elevated trace gas concentration; generating, by the at least one trace gas sensor, a first data and a second data packet of two or more data packets, where the first data packet and the second data packet comprise trace gas concentration data; receiving, by a processor of a control electronics, the first data packet and the second data packet; and processing, by the processor of the control electronics, the first data packet and the second data packet to include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected.

Additional method embodiments may further include: providing power to the at least one trace gas sensor via the control electronics. Additional method embodiments may further include: storing, by the processor of the control electronics, the first data packet in a memory of the control electronics; and transmitting, by the processor of the control electronics, the second data packet to a ground control system (GCS) via a transmitter of the control electronics.

Additional method embodiments may further include: receiving, by a processor of the ground control system, the second data packet. Additional method embodiments may further include: displaying, by the processor of the ground control system, at least one of: a map showing a location of the at least one trace gas sensor and a graph of the elevated trace gas concentration.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a system for integration of trace gas sensors onto an unmanned vehicle, according to one embodiment;

FIG. 2 depicts additional views of the unmanned vehicle of FIG. 1, according to one embodiment;

FIG. 3 depicts a top view of the system of FIG. 1, according to one embodiment;

FIG. 4A depicts a top view of a trace gas sensor, according to one embodiment;

FIG. 4B depicts a side view of the trace gas sensor of FIG. 4A, according to one embodiment;

FIG. 5 depicts an alternate system for integration of trace gas sensors onto an unmanned vehicle, according to one embodiment;

FIG. 6 depicts a block diagram of a Power Logger and GPS board (PLG) and a radio module, according to one embodiment;

FIG. 7 depicts a ground control station, according to one embodiment;

FIG. 8 depicts a top view of a trace gas sensor integrated into an unmanned vehicle with a D-sub electrical connector port, according to one embodiment;

FIG. 9 depicts a perspective view of a trace gas sensor integrated into an unmanned vehicle with an HB15 port, according to one embodiment;

FIG. 10 illustrates an example top-level functional block diagram of a flight pattern generation system for integration of trace gas sensors onto an unmanned vehicle, according to one embodiment, according to one embodiment;

FIG. 11 depicts a high-level flowchart of a method embodiment of integrating trace gas sensors onto an unmanned aerial vehicle, according to one embodiment;

FIG. 12 illustrates an example top-level functional block diagram of a computing device embodiment, according to one embodiment;

FIG. 13 shows a high-level block diagram and process of a computing system for implementing an embodiment of the system and process, according to one embodiment;

FIG. 14 shows a block diagram and process of an exemplary system in which an embodiment may be implemented, according to one embodiment;

FIG. 15 depicts a cloud-computing environment for implementing an embodiment of the system and process disclosed herein, according to one embodiment; and

FIG. 16 depicts a system for detecting trace gasses, according to one embodiment.

### DETAILED DESCRIPTION

The present system allows for a trace gas sensor to be attached to a mobile platform such that the trace gas sensor is not damaged during operation of the mobile platform and the mobile platform is not impeded in its movement. In some embodiments, the mobile platform may be an unmanned vehicle. In some embodiments, the mobile platform may be a quadrupedal robot. In other embodiments, the mobile platform may be a robot with wheels, tracks, or the like. In some embodiments, the trace gas sensor may obtain power from the mobile platform. The trace gas sensor may measure trace gas concentration, save relevant data, and provide a status of functionality. Data saved by the trace gas sensor may include a timestamp for when the measurement was taken, a sensor Time when measurement was taken, a trace gas concentration, a latitude and Longitude when measurement was taken (if available), and altitude when measurement was taken, a temperature when measurement was taken, and a pressure when measurement was taken.

Trace gas sensors are used to detect and quantify leaks of toxic gases, e.g., hydrogen disulfide, or environmentally damaging gases, e.g., methane and sulfur dioxide, in a variety of industrial and environmental contexts. Detection and quantification of these leaks are of interest to a variety of industrial operations, e.g., oil and gas, chemical production, and painting, as well as environmental regulators for assessing compliance and mitigating environmental and safety risks. The performance of trace gas sensors is typically described in terms of sensitivity, i.e., the lowest concentration a sensor can measure and the marginal change in concentration a sensor can measure, and specificity, i.e., how robust the concentration measurement is in a mixture of other gases. Laser-based gas detection techniques are capable of both highly sensitive and specific measurements. Laser-based measurements typically use a laser that emits at a wavelength of light that corresponds to an absorption transition of a chemical species of interest. This light is pitched across an empty space within a solid body, such as a cavity that contains the gas sample. The pitched light can either be at a fixed wavelength or it can be scanned in wavelength. A detector records how much light was transmitted across the cavity. Then, by using the Beer-Lambert relationship, which describes the transmission of light through a sample, i.e., gas in this case, as a function of sample composition and physical properties, e.g., composition, temperature, and pressure, the physical properties of the sample can be inferred. Laser-based trace gas sensors depend heavily on knowledge of the absorption spectrum of a molecule. The absorption spectrum is understood through a quantum-physics-based model that describes the allowable transitions in the energy level of a given molecule. These allowable changes in energy levels correspond to the wavelengths of light the molecule absorbs, and the selection of the energy level transition, or wavelength of light, to use in a trace gas sensor is key to determining the sensitivity and specificity of a sensor.

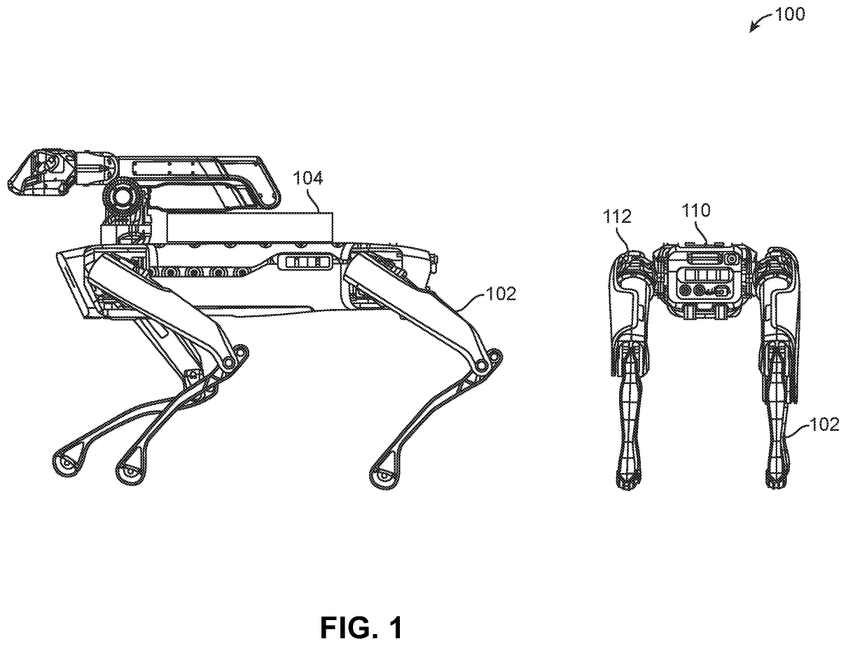

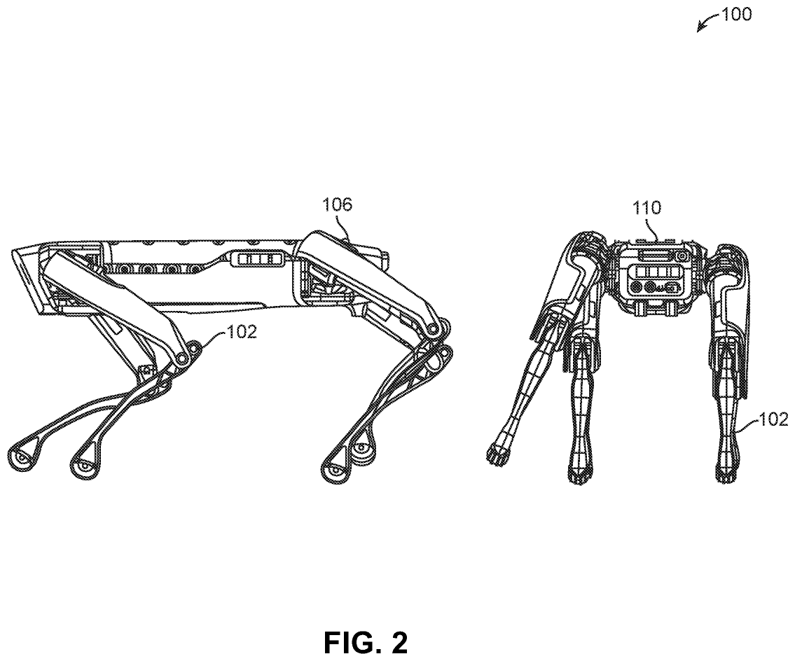

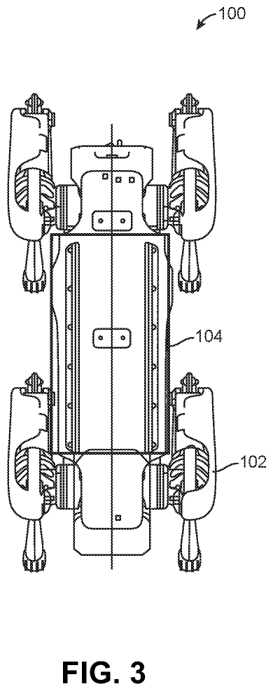

With respect to FIGS. 1-3, a system 100 allows for integration of one or more trace gas sensors 104 onto an unmanned vehicle 102, such as a ground robot. In one embodiment, the integration of the trace gas sensor 104 onto the unmanned vehicle 102 allow the trace gas sensor to obtain power from the unmanned vehicle 102, measure trace gas concentration, such as methane, in the ambient environment, save all relevant data, and provide status of the functionality of the system. In some embodiments, the sensor 104 may include an anemometer to measure wind speed and/or wind direction. Other sensors are possible and contemplated. The saved data of the one or more trace gas sensors 104 for when the measurement was taken may include a timestamp, the sensor time, the methane concentration, latitude and longitude, altitude, temperature, wind data such as wind speed and wind direction, and/or pressure.

The unmanned vehicle 102 may provide different power sources for the gas sensors 104. One power source may include an unregulated supply directly coming from a battery of the unmanned vehicle 102. The maximum voltage of the power source may peak as the unmanned vehicle 102 may contain a regenerative system to prolong the battery life. In one embodiment, the connector type may be a female DB25. A second power source may be used in some embodiments. The second power source may include a regulated supply via a prototype adapter that may mount on back rails of the unmanned vehicle 102. In one embodiment, the connector type may be a female HD15.

In one embodiment, power integration may be achieved via a female DB25 connector. An isolated DC/DC converter may be integrated.

In one embodiment, the one or more trace gas sensors 104 may be mounted to a top side of the unmanned vehicle 102, such as at mounting location 106. Mounting within a designated area may provide for avoiding intersection with legs, or other moving parts, of the unmanned vehicle 102. The unmanned vehicle 102 may include M5 clearance holes that may be spaced 150 mm apart. The unmanned vehicle 102 may further include Short Motor and Payload safety on the DB25 connector. The unmanned vehicle 102 may also include a stop mechanism using a motor interlock. The unmanned vehicle 102 may also be configured for USB connections and wireless connections, such as WiFi for streaming of data gathered by the trace gas sensors.

In some embodiments, the one or more trace gas sensors 104 may be mounted within a body 110 width so as to avoid impacting one or more moving portions of the unmanned vehicle 102. In other embodiments, the one or more trace gas sensors 104 may extend beyond a body 110 width so long as the shape does not unduly impact mobility of one or more moveable portions 112 of the unmanned vehicle 102. For example, the one or more trace gas sensors 104 may be mounted on a top portion of an unmanned vehicle having articulated limbs as moveable portions 112 such that the moveable portions 112 are not impeded by the placement of the one or more trace gas sensors 104. In some embodiments, the unmanned vehicle 102 may have articulated limbs so as to be able to traverse uneven terrain. In other embodiments, the unmanned vehicle 102 may have wheels, tracks, a rolling mechanism, or other methods of transport over even and uneven terrain. In some embodiments, the one or more trace gas sensors 104 may be connected proximate a center of gravity of the unmanned vehicle 102, such as a midpoint between hips of the unmanned vehicle 102. In some embodiments, the one or more trace gas sensors 104 may be mounted in a location so as to be protected during use, such as against a flat portion where the one or more trace gas sensors 104 are unlikely to be damaged during movement of the unmanned vehicle 102.

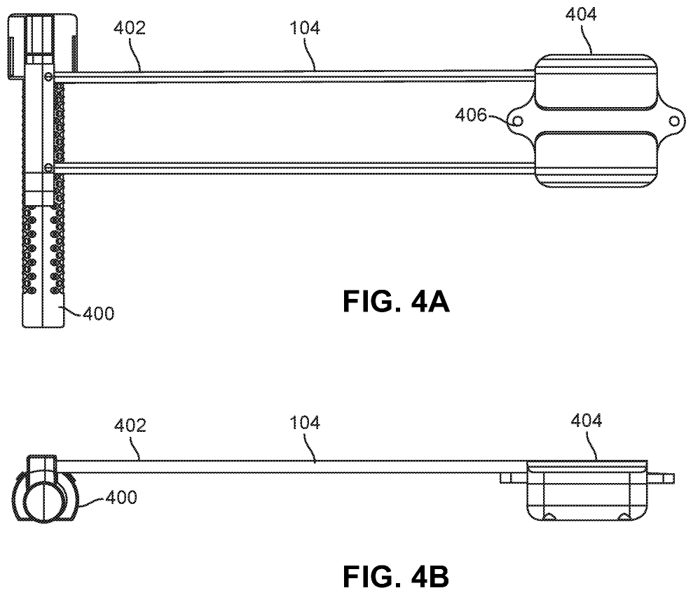

With respect to FIGS. 4A and 4B, a top perspective view and a side perspective view of a trace gas sensor 104 are shown, respectively. The trace gas sensor 104 may be configured to measure ambient trace gas concentrations of at least one of the following gasses: methane, ethane, propane, butane, and/or natural gas. The trace gas sensor 104 may include a cover 400 protecting a gas sensor disposed inside the cover 400, one or more extenders 402, control electronics 404, and/or one or more mounting attachments 406. The cover may include air holes for allowing air into a trace gas sensor for detecting trace gas concentrations while protecting the trace gas sensor from impacts, dust, and the like. The extenders 402 may include one or more rods to place the sensor and cover 400 distal from the mounting attachments 406. The extenders 402 may be used to allow the sensor to be located away from an unmanned vehicle for more accurate ambient readings of trace gas concentrations. The control electronics 404 may include a processor, addressable memory, a transmitter, a receiver, a transceiver, and/or a power source. The control electronics 404 may be in contact with the sensor. The mounting attachments 406 may allow the gas sensor 104 to be attached to an unmanned vehicle, such as via screws, nuts and bolts, or the like. While one configuration is shown, other gas sensor configurations for mounting to an unmanned vehicle are possible and contemplated.

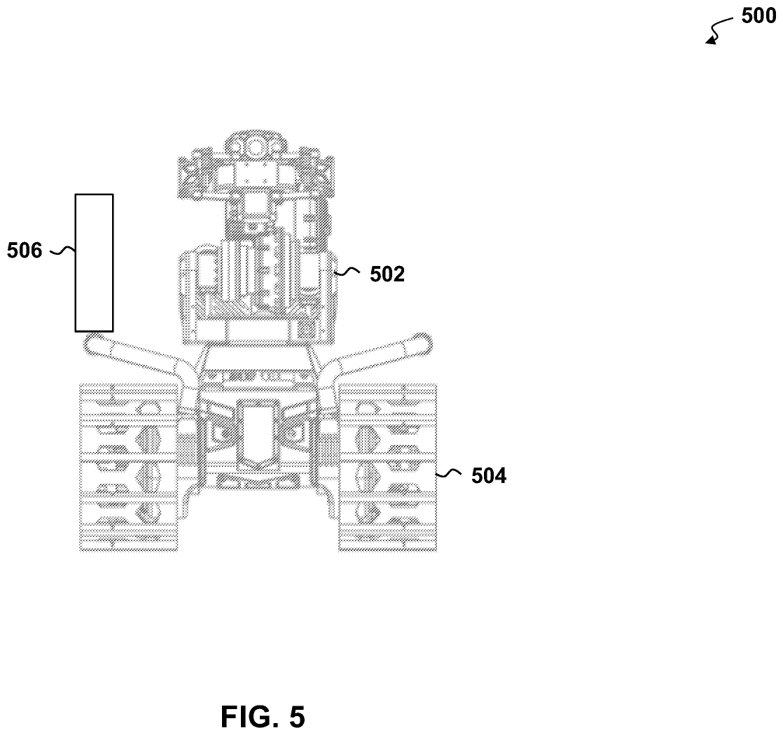

FIG. 5 depicts an alternate system 500 for integration of trace gas sensors onto an unmanned vehicle, according to one embodiment. The system 500 allows for integration of one or more trace gas sensors 506 onto an unmanned vehicle 502, such as a ground robot with tracks 504 for movement. In one embodiment, the integration of the trace gas sensor 506 onto the unmanned vehicle 502 allows the trace gas sensor to obtain power from the unmanned vehicle 502, measure trace gas concentration, such as methane, in the ambient environment, save all relevant data, and provide status of the functionality of the system. The saved data of the one or more trace gas sensors 506 for when the measurement was taken may include a timestamp, the sensor time, the methane concentration, latitude and longitude, altitude, temperature, wind data, and/or pressure. The sensor 506 may be located so as to not interfere with movement of the unmanned vehicle 502, such as by not interfering with the movement of the tracks 504.

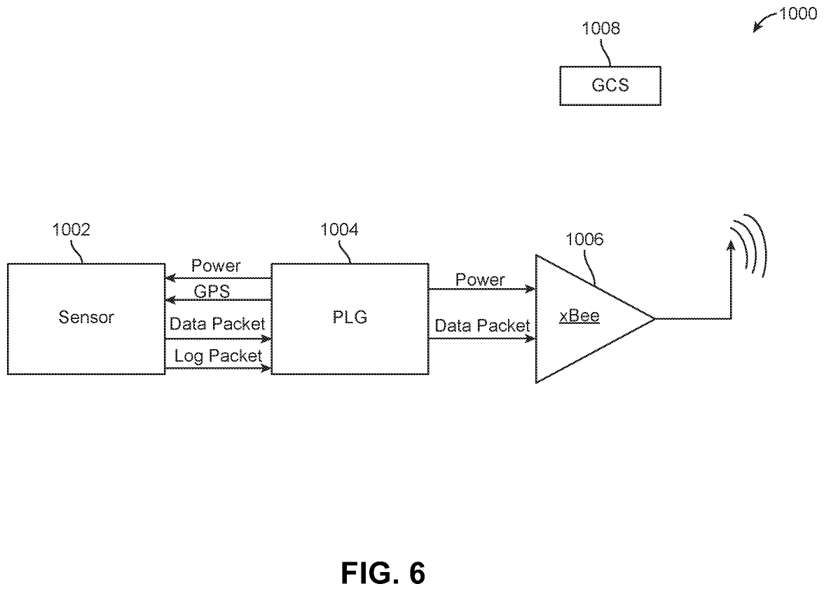

FIG. 6 shows a block diagram 1000 of the integration of a PLG (Power Logger and GPS board), and a programmable module, such as an xBee. A cable may connect the unmanned vehicle to the one or more trace gas sensors. More specifically, the PLG may be integrated into the trace gas sensors to provide adequate voltage regulation to the trace gas sensors as well as to have access to GPS information and a logging system. The PLG may replace a DC/DC converter providing power to the sensor as well GPS data. In one embodiment, the one or more trace gas sensors 1002 may process trace gas concentrations and when an elevated trace gas concentration is detected, two data packets may be created by the sensor 1002.

A first data packet may be sent to the logging system which is present on the PLG 1004 described above. The PLG 1004 may have a micro SD card, or other addressable memory, on board. In one embodiment, a user may remove the micro SD card and replace it with a new micro SD card if the user wants to keep the original micro SD card. A second packet, such as a log packet, may be passed over to the xBee 1006 in order to send the information to a Ground Control Station (GCS) 1008.

The GCS 1008 may be used to observe proper operation of the trace gas sensors 1002 from a distance. In one embodiment, the xBee 1006 is a a radio transmitter. In some embodiments, the GCS 1008 may be modified to receive and process data packets as well as to auto connect to the xBee 1006 automatically. In some embodiments, the sensor 1002 and/or xBee 1006 may receive power from the PLG 1004. In other embodiments, the sensor 1002 and/or the xBee 1006 may receive power from a secondary power source. While an xBee 1006 is disclosed, other compatible wireless connectivity modules are possible and contemplated.

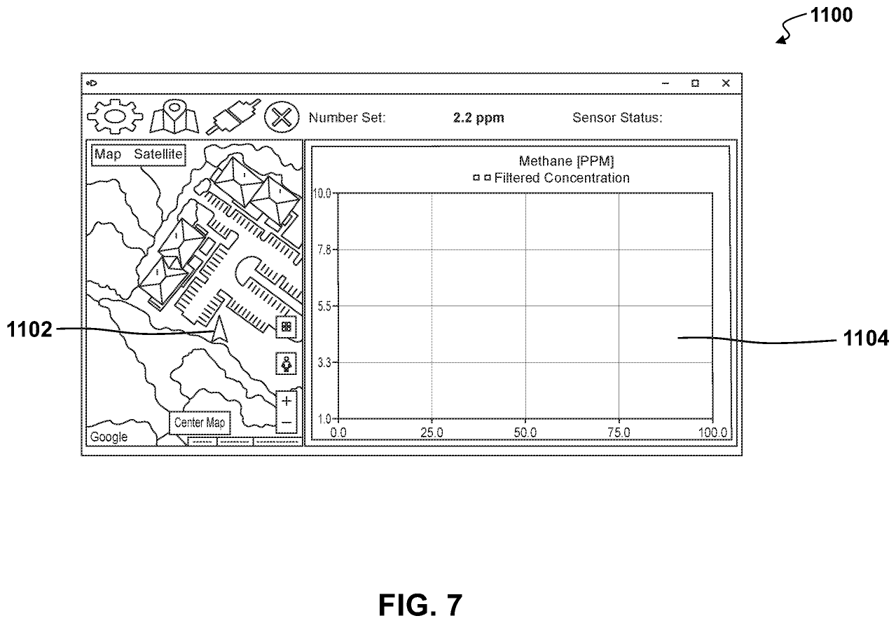

With respect to FIG. 7, a graph 1100 of a GCS, such as the GCS described above is illustrated. A trace gas sensor location 1102 is shown in the left panel and the associated measured trace gas concentration detected by the gas sensor is plotted in the graph 1104 on the right panel. Other gas concentrations may be measured and plotted in other embodiments. Using the map on the left, a user may see the location of the unmanned vehicle with the mounted gas sensor (see the system of FIG. 1). In the graph on the right panel, the corresponding real-time trace gas concentrations detected by the trace gas sensor may be displayed. In one embodiment, the GCS may save data locally in order to add another layer of backup for the data obtained from the trace gas sensor. In other embodiments, the GCS may transfer data to a cloud server for further processing.

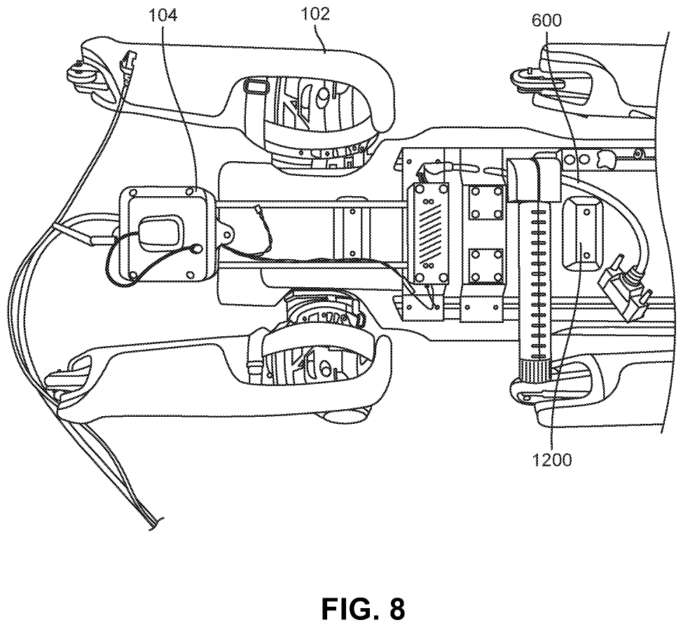

With respect to FIG. 8, a trace gas sensor 104, such as a methane gas sensor described above is shown integrated into an unmanned vehicle 102 with a DB25 port 1200 connected to a DB25 cable 600. Other ports and cables are possible and contemplated.

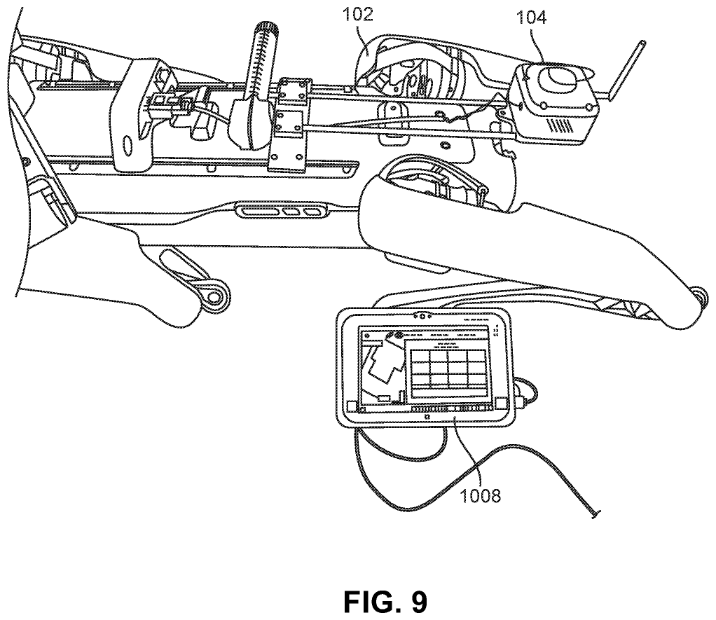

With respect to FIG. 9, the gas sensor 104 is shown integrated into the unmanned vehicle 102 with an HB15 port. The HB15 port may provide for wireless data streaming to the GCS 1008. Other ports are possible and contemplated. In one embodiment, the gas sensor draws may power from an adapter. The sensor may also send data wirelessly to a wireless computing device, such as a tablet or GCS 1008. At the wireless computing device, a user may reference and observe the proper operation of the gas sensor 104.

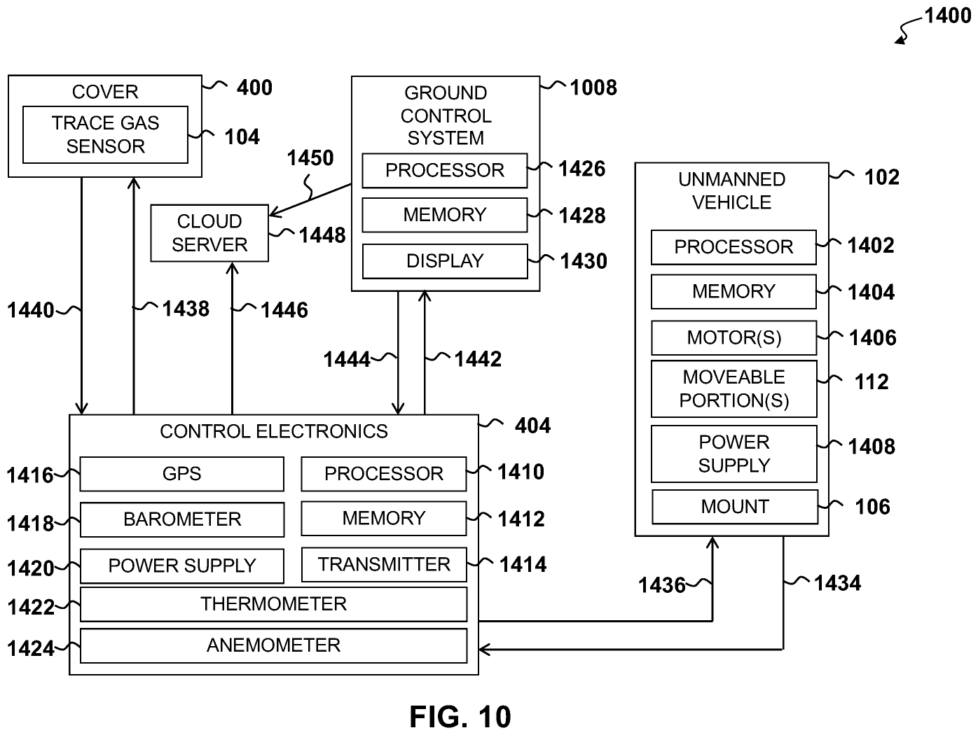

FIG. 10 illustrates an example top-level functional block diagram of a flight pattern generation system 1400 for integration of trace gas sensors 104 onto an unmanned vehicle 102, according to one embodiment, according to one embodiment. The system 1400 may include an unmanned vehicle 102, at least one trace gas sensor 104, control electronics 404, and a ground control system 1008.

The unmanned vehicle 102 may be a land vehicle such as a quadrupedal robot. The unmanned vehicle 102 may have a processor 1402 with addressable memory 1404, one or more motors 1406 controlling one or more moveable portions 112, a power supply 1408, and one or more mounts 106. The moveable portions 112 may include one or more legs, such as four legs in a quadrupedal robot. In other embodiments, the moveable portions 112 may include wheels, treads, tracks, or the like. The power supply 1408 may be a battery.

The power supply 1408 of the unmanned vehicle 102 may be used to power 1434 the control electronics 404 and/or the at least one trace gas sensor 104. The control electronics 404 may send data 1436 to the unmanned vehicle 102 in some embodiments. The unmanned vehicle 102 may send power and/or data 1434 to the control electronics 404 in some embodiments.

The mount 106 may be used to connect the at least one trace gas sensor 104 and/or control electronics 404 to the unmanned vehicle 102. In one embodiment, the mount 106 may include spaced apertures for receiving one or more screws, bolts, or the like. Other mounts 106 are possible and contemplated. The mount 106 may secure the at least one trace gas sensor 104 and/or control electronics 404 to the unmanned vehicle 102 without impeding movement of the moveable portions 112 of the unmanned vehicle 102. In some embodiments, the at least one trace gas sensor 104 may be positioned so that at least a portion of the trace gas sensor 104 is outside of a body of the unmanned vehicle 102 so as to provide more accurate trace gas readings.

The at least one trace gas sensor 104 may include a cover 400 to protect the at least one trace gas sensor 104, as shown in FIGS. 4A-4B. The trace gas sensor 104 may measure ambient gas concentrations and detect elevated trace gas concentrations. The trace gas sensor 104 may receive power 1438 from the control electronics 404. The trace gas sensor 104 may generate a first data and a second data packet of two or more data packets. The first data packet and the second data packet may include the trace gas concentration data from the trace gas sensor 104. The trace gas sensor 104 may send 1440 the first data packet and the second data packet to the control electronics.

The control electronics 404 may include a processor 1410 having addressable memory 1412, a transmitter 1414, a global positioning system (GPS) 1416, a barometer or other pressure sensor 1418, a power supply 1420, a thermometer or other temperature sensor 1422, and an anemometer 1424. The anemometer 1424 may provide wind data. The wind data may include wind speed and/or wind direction. The GPS 1416 may be another positioning system in some embodiments. The power supply 1420 may not be present in some embodiments where the control electronics 404 receive power from the unmanned vehicle 102.

The processor 1410 of the control electronics 404 may process the first data packet and the second data packet received 1440 from the trace gas sensor 104 to include related data from one or more sensors, such as the GPS 1416, barometer 1418, thermometer 1422, and/or anemometer 1424. The data from one or more sensors may include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected. In some embodiments, the data from the one or more sensors may be received by the control electronics 404 from a third party source, such as a remote temperature station. In some embodiments, the processor 1410 of the control electronics 404 may determine an altitude based on a latitude and longitude from the GPS 1416 corresponding to a map having altitude measurements. Other sources of additional data are possible and contemplated.

The processor 1410 of the control electronics 404 may store the first data packet in the memory 1412. In some embodiments, the stored data may be stored on a removable memory device, such as a micro SD card that may be removed by a user for transferring data to another computing device, cloud server, or the like.

The processor 1410 of the control electronics 404 may transmit 1442, via the transmitter 1414, the second data packet to the GCS 1008. In some embodiments, the transmitter 1414 may be a compatible wireless connectivity modules, such as an xBee. In some embodiments, the transmitter 1414 may be a transceiver to receive 1444 data from a ground control system 1008.

The processor 1410 of the control electronics 404 may store one or more data packet locally, such as in memory 1412. The processor 1410 of the control electronics 404 may also transmit 1442, via the transmitter 1414, one or more data packets to the GCS 1008. The processor 1410 of the control electronics 404 may also transmit 1442, via the transmitter 1414, one or more data packets to cloud server 1448. In some embodiments, the one or more data packets may be sent between the local storage, the GCS 1008, and the cloud server 1448. For example, a data packet could be transmitted 1442 to the GCS 1008, stored in the memory 1428 of the GCS 1008, and/or transmitted 1450 to the cloud server via the GCS 1008. In one embodiment, only one data packet may be generated. In other embodiments, two or more data packets may be generated. Data packets may be stored in memory and/or streamed to the GCS 1008 and/or cloud server 1448 in real time or near real time in some embodiments. The streaming or transmitting of the data packets may be via Global System for Mobile Communications (GSM); text messaging; an Internet endpoint; a wireless network protocol such as Wi-Fi or Bluetooth; and the like.

The GCS 1008 may include a processor 1426 having addressable memory 1428 and a display 1430. The processor 1426 of the GCS 1008 may receive 1442 the second data packet from the control electronics 404. The GCS 1008 may display at least one of: a map showing a location of the at least one trace gas sensor and a graph of the elevated trace gas concentration, as shown in FIG. 7. An operator of the GCS 1008 may adjust a movement of the unmanned vehicle 102 based on elevated trace gas readings. For example, an operator may move the unmanned vehicle 102 about a potential trace gas source to determine whether the potential trace gas source is emitting trace gas at elevated levels, such as via a leak. If a leak is detected, the operator may be able to take corrective actions to stop the leak of trace gas.

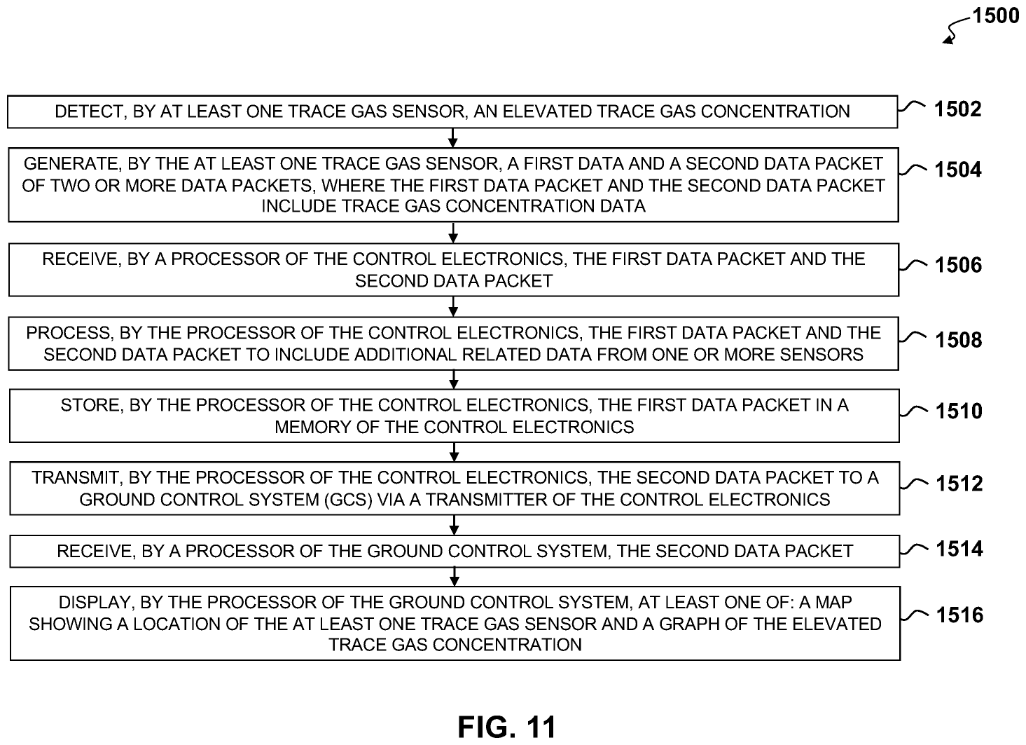

FIG. 11 depicts a high-level flowchart of a method embodiment 1500 of integrating trace gas sensors onto an unmanned aerial vehicle, according to one embodiment. The method 1500 may include providing power to at least one trace gas sensor via a control electronics. The control electronics may receive power from the unmanned vehicle in some embodiments. In other embodiments, the at least one trace gas sensor may receive power from the unmanned vehicle. In other embodiments, the at least one trace gas sensor may receive power from a separate power source.

The method 1500 may also include detecting, by the at least one trace gas sensor, an elevated trace gas concentration (step 1502). The method 1500 may then include generating, by the at least one trace gas sensor, a first data and a second data packet of two or more data packets, where the first data packet and the second data packet comprise trace gas concentration data (step 1504). The method 1500 may then include receiving, by a processor of the control electronics, the first data packet and the second data packet (step 1506).

The method 1500 may then include processing, by the processor of the control electronics, the first data packet and the second data packet to include related data from one or more sensors (step 1508). The data from one or more sensors may include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected.

The method 1500 may then include storing, by the processor of the control electronics, the first data packet in a memory of the control electronics (step 1510). In some embodiments, the stored data may be stored on a removable memory device, such as a micro SD card that may be removed by a user for transferring data to another computing device, cloud server, or the like. The method 1500 may then include transmitting, by the processor of the control electronics, the second data packet to a ground control system (GCS) via a transmitter of the control electronics (step 1512). In some embodiments, the transmitter may be a compatible wireless connectivity modules, such as an xBee. The method 1500 may then include receiving, by a processor of the ground control system, the second data packet (step 1514). The method 1500 may then include displaying, by the processor of the ground control system, at least one of: a map showing a location of the at least one trace gas sensor and a graph of the elevated trace gas concentration (step 1516).

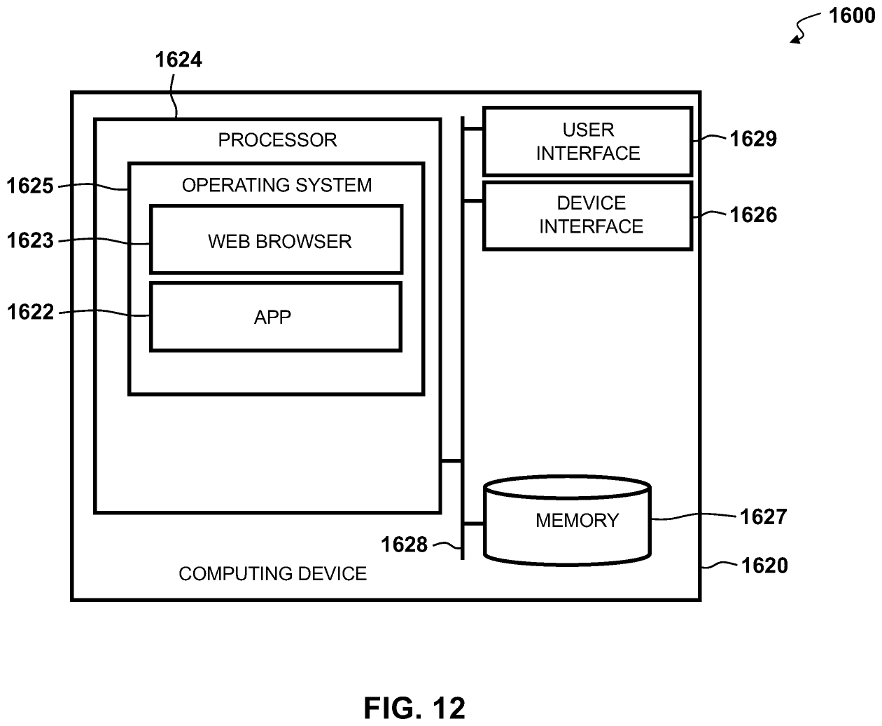

FIG. 12 illustrates an example of a top-level functional block diagram of a computing device embodiment 1600. The example operating environment is shown as a computing device 1620 comprising a processor 1624, such as a central processing unit (CPU), addressable memory 1627, an external device interface 1626, e.g., an optional universal serial bus port and related processing, and/or an Ethernet port and related processing, and an optional user interface 1629, e.g., an array of status lights and one or more toggle switches, and/or a display, and/or a keyboard and/or a pointer-mouse system and/or a touch screen. Optionally, the addressable memory may, for example, be: flash memory, eprom, and/or a disk drive or other hard drive. These elements may be in communication with one another via a data bus 1628. In some embodiments, via an operating system 1625 such as one supporting a web browser 1623 and applications 1622, the processor 1624 may be configured to execute steps of a process establishing a communication channel and processing according to the embodiments described above.

System embodiments include computing devices such as a server computing device, a buyer computing device, and a seller computing device, each comprising a processor and addressable memory and in electronic communication with each other. The embodiments provide a server computing device that may be configured to: register one or more buyer computing devices and associate each buyer computing device with a buyer profile; register one or more seller computing devices and associate each seller computing device with a seller profile; determine search results of one or more registered buyer computing devices matching one or more buyer criteria via a seller search component. The service computing device may then transmit a message from the registered seller computing device to a registered buyer computing device from the determined search results and provide access to the registered buyer computing device of a property from the one or more properties of the registered seller via a remote access component based on the transmitted message and the associated buyer computing device; and track movement of the registered buyer computing device in the accessed property via a viewer tracking component. Accordingly, the system may facilitate the tracking of buyers by the system and sellers once they are on the property and aid in the seller's search for finding buyers for their property. The figures described below provide more details about the implementation of the devices and how they may interact with each other using the disclosed technology.

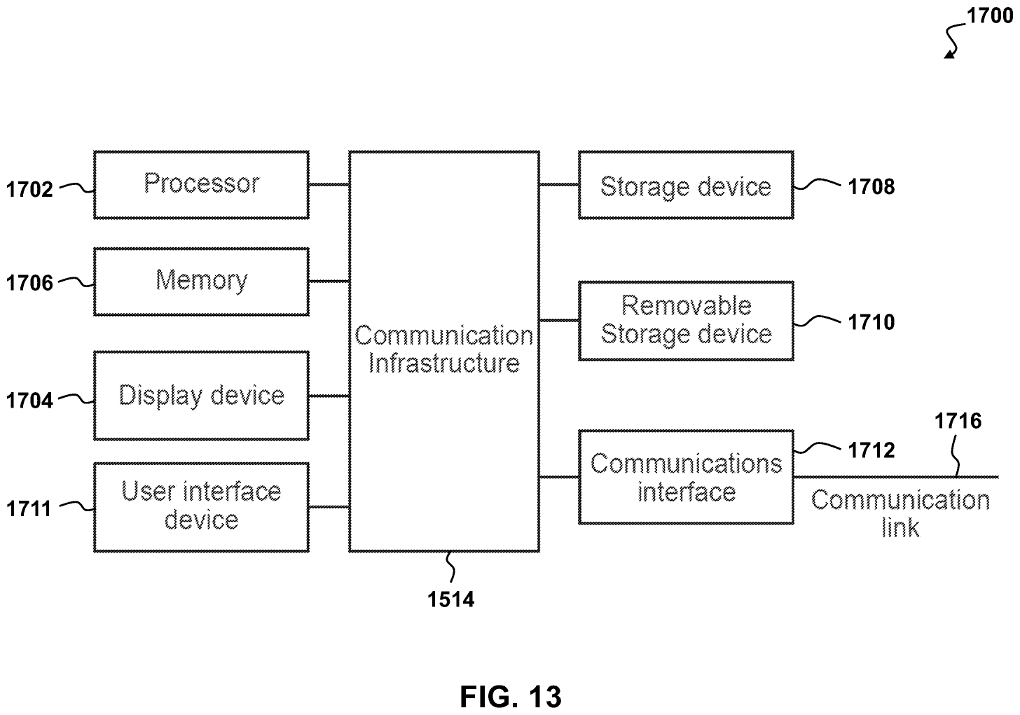

FIG. 13 is a high-level block diagram 1700 showing a computing system comprising a computer system useful for implementing an embodiment of the system and process, disclosed herein. Embodiments of the system may be implemented in different computing environments. The computer system includes one or more processors 1702, and can further include an electronic display device 1704 (e.g., for displaying graphics, text, and other data), a main memory 1706 (e.g., random access memory (RAM)), storage device 1708, a removable storage device 1710 (e.g., removable storage drive, a removable memory module, a magnetic tape drive, an optical disk drive, a computer readable medium having stored therein computer software and/or data), user interface device 1711 (e.g., keyboard, touch screen, keypad, pointing device), and a communication interface 1712 (e.g., modem, a network interface (such as an Ethernet card), a communications port, or a PCMCIA slot and card). The communication interface 1712 allows software and data to be transferred between the computer system and external devices. The system further includes a communications infrastructure 1714 (e.g., a communications bus, cross-over bar, or network) to which the aforementioned devices/modules are connected as shown.

Information transferred via communications interface 1714 may be in the form of signals such as electronic, electromagnetic, optical, or other signals capable of being received by communications interface 1714, via a communication link 1716 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 1712. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

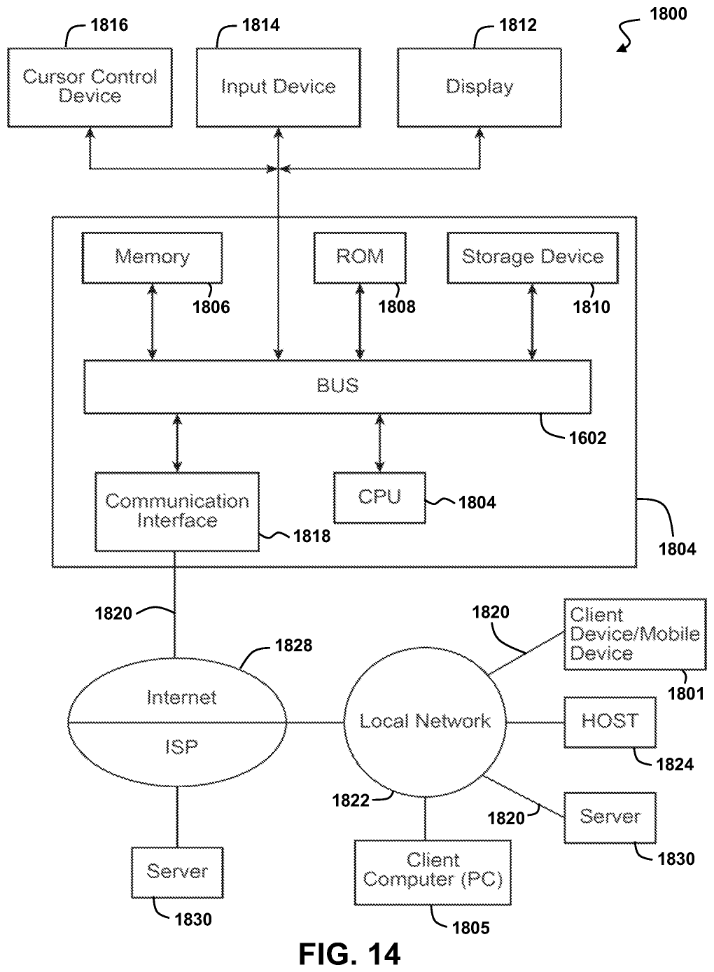

FIG. 14 shows a block diagram of an example system 1800 in which an embodiment may be implemented. The system 1800 includes one or more client devices 1801 such as consumer electronics devices, connected to one or more server computing systems 1830. A server 1830 includes a bus 1802 or other communication mechanism for communicating information, and a processor (CPU) 1804 coupled with the bus 1802 for processing information. The server 1830 also includes a main memory 1806, such as a random access memory (RAM) or other dynamic storage device, coupled to the bus 1802 for storing information and instructions to be executed by the processor 1804. The main memory 1806 also may be used for storing temporary variables or other intermediate information during execution or instructions to be executed by the processor 1804. The server computer system 1830 further includes a read only memory (ROM) 1808 or other static storage device coupled to the bus 1802 for storing static information and instructions for the processor 1804. A storage device 1810, such as a magnetic disk or optical disk, is provided and coupled to the bus 1802 for storing information and instructions. The bus 1802 may contain, for example, thirty-two address lines for addressing video memory or main memory 1806. The bus 1802 can also include, for example, a 32-bit data bus for transferring data between and among the components, such as the CPU 1804, the main memory 1806, video memory and the storage 1810. Alternatively, multiplex data/address lines may be used instead of separate data and address lines.

The server 1830 may be coupled via the bus 1802 to a display 1812 for displaying information to a computer user. An input device 1814, including alphanumeric and other keys, is coupled to the bus 1802 for communicating information and command selections to the processor 1804. Another type or user input device comprises cursor control 1816, such as a mouse, a trackball, or cursor direction keys for communicating direction information and command selections to the processor 1804 and for controlling cursor movement on the display 1812.

According to one embodiment, the functions are performed by the processor 1804 executing one or more sequences of one or more instructions contained in the main memory 1806. Such instructions may be read into the main memory 1806 from another computer-readable medium, such as the storage device 1810. Execution of the sequences of instructions contained in the main memory 1806 causes the processor 1804 to perform the process steps described herein. One or more processors in a multi-processing arrangement may also be employed to execute the sequences of instructions contained in the main memory 1806. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1804 for execution. Such a medium may take many forms, including but not limited to, non-volatile media, volatile media, and transmission media. Non-volatile media includes, for example, optical or magnetic disks, such as the storage device 1810. Volatile media includes dynamic memory, such as the main memory 1806. Transmission media includes coaxial cables, copper wire and fiber optics, including the wires that comprise the bus 1802. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1804 for execution. For example, the instructions may initially be carried on a magnetic disk of a remote computer. The remote computer can load the instructions into its dynamic memory and send the instructions over a telephone line using a modem. A modem local to the server 1830 can receive the data on the telephone line and use an infrared transmitter to convert the data to an infrared signal. An infrared detector coupled to the bus 1802 can receive the data carried in the infrared signal and place the data on the bus 1802. The bus 1802 carries the data to the main memory 1806, from which the processor 1804 retrieves and executes the instructions. The instructions received from the main memory 1806 may optionally be stored on the storage device 1810 either before or after execution by the processor 1804.

The server 1830 also includes a communication interface 1818 coupled to the bus 1802. The communication interface 1818 provides a two-way data communication coupling to a network link 1820 that is connected to the world wide packet data communication network now commonly referred to as the Internet 1828. The Internet 1828 uses electrical, electromagnetic or optical signals that carry digital data streams. The signals through the various networks and the signals on the network link 1820 and through the communication interface 1818, which carry the digital data to and from the server 1830, are exemplary forms or carrier waves transporting the information.

In another embodiment of the server 1830, interface 1818 is connected to a network 1822 via a communication link 1820. For example, the communication interface 1818 may be an integrated services digital network (ISDN) card or a modem to provide a data communication connection to a corresponding type of telephone line, which can comprise part of the network link 1820. As another example, the communication interface 1818 may be a local area network (LAN) card to provide a data communication connection to a compatible LAN. Wireless links may also be implemented. In any such implementation, the communication interface 1818 sends and receives electrical electromagnetic or optical signals that carry digital data streams representing various types of information.

The network link 1820 typically provides data communication through one or more networks to other data devices. For example, the network link 1820 may provide a connection through the local network 1822 to a host computer 1824 or to data equipment operated by an Internet Service Provider (ISP). The ISP in turn provides data communication services through the Internet 1828. The local network 1822 and the Internet 1828 both use electrical, electromagnetic or optical signals that carry digital data streams. The signals through the various networks and the signals on the network link 1820 and through the communication interface 1818, which carry the digital data to and from the server 1830, are exemplary forms or carrier waves transporting the information.

The server 1830 can send/receive messages and data, including e-mail, program code, through the network, the network link 1820 and the communication interface 1818. Further, the communication interface 1818 can comprise a USB/Tuner and the network link 1820 may be an antenna or cable for connecting the server 1830 to a cable provider, satellite provider or other terrestrial transmission system for receiving messages, data and program code from another source.

The example versions of the embodiments described herein may be implemented as logical operations in a distributed processing system such as the system 1800 including the servers 1830. The logical operations of the embodiments may be implemented as a sequence of steps executing in the server 1830, and as interconnected machine modules within the system 1800. The implementation is a matter of choice and can depend on performance of the system 1800 implementing the embodiments. As such, the logical operations constituting said example versions of the embodiments are referred to for e.g., as operations, steps or modules.

Similar to a server 1830 described above, a client device 1801 can include a processor, memory, storage device, display, input device and communication interface (e.g., e-mail interface) for connecting the client device to the Internet 1828, the ISP, or LAN 1822, for communication with the servers 1830.

The system 1800 can further include computers (e.g., personal computers, computing nodes) 1805 operating in the same manner as client devices 1801, where a user can utilize one or more computers 1805 to manage data in the server 1830.

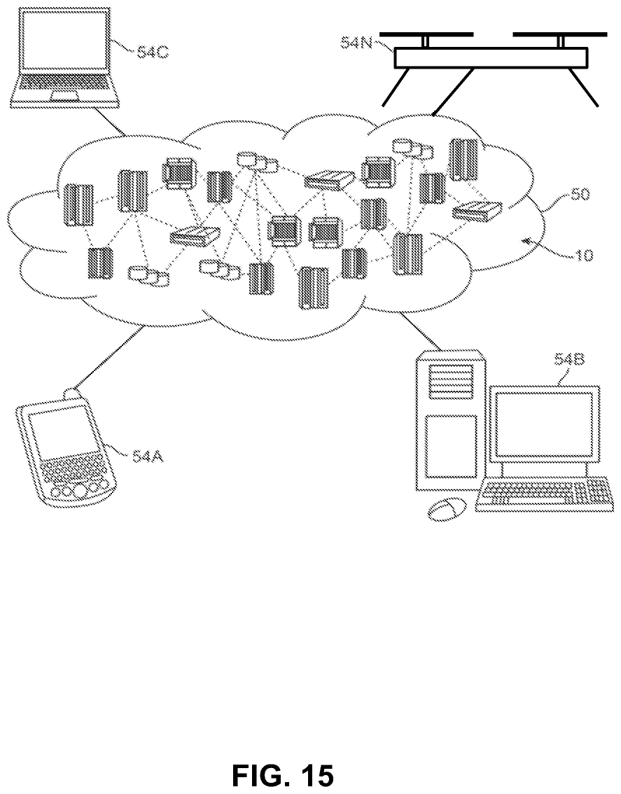

Referring now to FIG. 15, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or unmanned aerial system (UAS) 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 15 are intended to be illustrative only and that computing nodes 10 and cloud computing environment 50 can communicate with any type of computerized device over any type of network and/or network addressable connection (e.g., using a web browser).

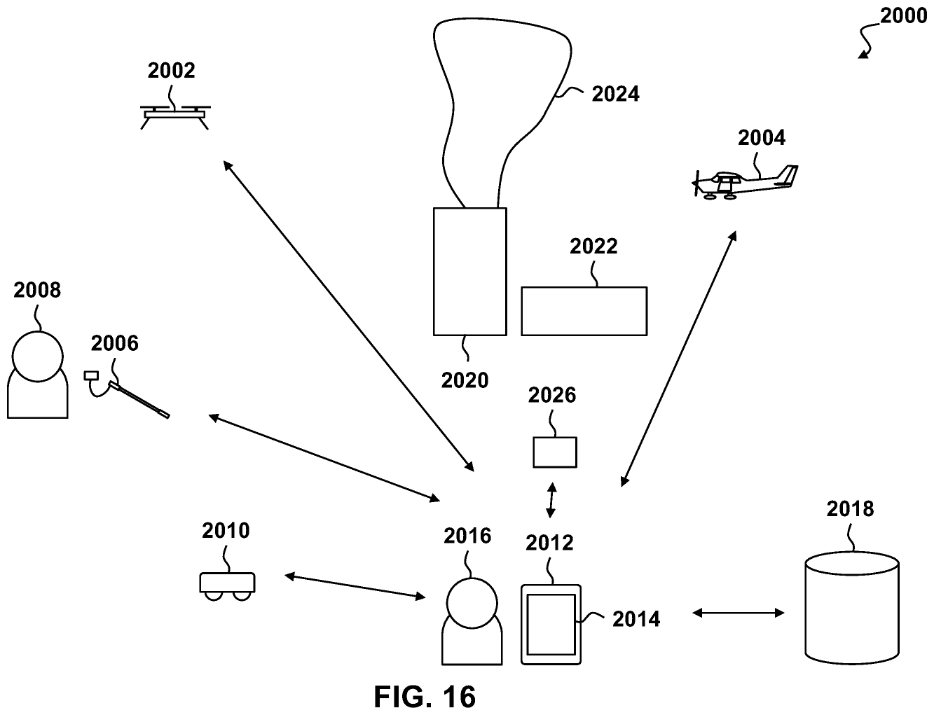

FIG. 16 depicts a system 2000 for detecting trace gasses, according to one embodiment. The system may include one or more trace gas sensors located in one or more vehicles 2002, 2004, 2006, 2010. The one or more trace gas sensors may detect elevated trace gas concentrations from one or more potential gas sources 2020, 2022, such as a holding tank, pipeline, or the like. The potential gas sources 2020, 2022 may be part of a large facility, a small facility, or any location. The potential gas sources 2020, 2022 may be clustered and/or disposed distal from one another. The one or more trace gas sensors may be used to detect and quantify leaks of toxic gases, e.g., hydrogen disulfide, or environmentally damaging gases, e.g., methane, sulfur dioxide) in a variety of industrial and environmental contexts. Detection and quantification of these leaks are of interest to a variety of industrial operations, such as oil and gas, chemical production, and painting. Detection and quantification of leaks is also of value to environmental regulators for assessing compliance and for mitigating environmental and safety risks. In some embodiments, the at least one trace gas sensor may be configured to detect methane. In other embodiments, the at least one trace gas sensor may be configured to detect sulfur oxide, such as SO, SO2, SO3, S7O2, S6O2, S2O2, and the like. A trace gas leak 2024 may be present in a potential gas source 2020. The one or more trace gas sensors may be used to identify the trace gas leak 2024 and/or the source 2020 of the trace gas leak 2024 so that corrective action may be taken.

The one or more vehicles 2002, 2004, 2006, 2010 may include an unmanned aerial vehicle (UAV) 2002, an aerial vehicle 2004, a handheld device 2006, and a ground vehicle 2010. In some embodiments, the UAV 2002 may be a quadcopter or other device capable of hovering, making sharp turns, and the like. In other embodiments, the UAV 2002 may be a winged aerial vehicle capable of extended flight time between missions. The UAV 2002 may be autonomous or semi-autonomous in some embodiments. In other embodiments, the UAV 2002 may be manually controlled by a user. The aerial vehicle 2004 may be a manned vehicle in some embodiments. The handheld device 2006 may be any device having one or more trace gas sensors operated by a user 2008. In one embodiment, the handheld device 2006 may have an extension for keeping the one or more trace gas sensors at a distance from the user 2008. The ground vehicle 2010 may have wheels, tracks, and/or treads in one embodiment. In other embodiments, the ground vehicle 2010 may be a legged robot. In some embodiments, the ground vehicle 2010 may be used as a base station for one or more UAVs 2002. In some embodiments, one or more aerial devices, such as the UAV 2002, a balloon, or the like, may be tethered to the ground vehicle 2010. In some embodiments, one or more trace gas sensors may be located in one or more stationary monitoring devices 2026. The one or more stationary monitoring devices may be located proximate one or more potential gas sources 2020, 2022. In some embodiments, the one or more stationary monitoring devices may be relocated.

The one or more vehicles 2002, 2004, 2006, 2010 and/or stationary monitoring devices 2026 may transmit data including trace gas data to a ground control station (GCS) 2012. The GCS may include a display 2014 for displaying the trace gas concentrations to a GCS user 2016. The GCS user 2016 may be able to take corrective action if a gas leak 2024 is detected, such as by ordering a repair of the source 2020 of the trace gas leak. The GCS user 2016 may be able to control movement of the one or more vehicles 2002, 2004, 2006, 2010 in order to confirm a presence of a trace gas leak in some embodiments.

In some embodiments, the GCS 2012 may transmit data to a cloud server 2018. In some embodiments, the cloud server 2018 may perform additional processing on the data. In some embodiments, the cloud server 2018 may provide third party data to the GCS 2012, such as wind speed, temperature, pressure, weather data, or the like.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A system comprising:
at least one trace gas sensor configured to measure trace gas concentrations, wherein the at least one trace gas sensor is configured to generate a first data and a second data packet of two or more data packets, wherein the first data packet and the second data packet comprise trace gas concentration data;
a control electronics in communication with the at least one trace gas sensor, wherein the control electronics comprises a processor configured to:
process the first data packet and the second data packet to include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected;
store, the first data packet in a memory of the control electronics; and
transmit the second data packet to a ground control system (GCS) via a transmitter of the control electronics;
an unmanned vehicle, wherein the at least one trace gas sensor is attached to the unmanned vehicle such that the at least one trace gas sensor does not impair movement of the unmanned vehicle;
wherein the GCS is configured to display a location of the unmanned vehicle and a corresponding real-time trace gas concentration detected by the at least one trace gas sensor.

2. The system of claim 1, wherein the unmanned vehicle is a quadrupedal robot.

3. The system of claim 1, wherein the unmanned vehicle is a ground robot.

4. The system of claim 1, wherein the at least one trace gas sensor extends beyond a body width of the unmanned vehicle.

5. The system of claim 1, wherein the at least one trace gas sensor is configured to measure trace gas concentrations of at least one of: methane, ethane, propane, butane, and natural gas.

6. The system of claim 1, wherein the at least one trace gas sensor further comprises:
a cover configured to protect a sensor of the trace gas sensor, wherein the cover comprises one or more air holes for allowing airflow into the sensor while protecting the sensor from impacts and dust;
one or more mounting attachments for connecting the at least one trace gas sensor to the unmanned vehicle; and
one or more extenders configured to place the sensor distal from the one or more mounting attachments.

7. The system of claim 1, wherein the memory of the control electronics comprises a micro SD card.

8. The system of claim 1, wherein the GCS is further configured to transmit the received second data packet to a cloud server.

9. The system of claim 1, wherein the GCS is further configured to store the second data packet in a memory of the GCS.

10. The system of claim 1, further comprising: an adapter, wherein the adapter provides power to the at least one trace gas sensor and the control electronics from a power supply of the unmanned vehicle.

11. The system of claim 1, further comprising: an adapter, wherein the adapter provides data for at least one: the timestamp for when the elevated trace gas concentration was detected, the latitude and longitude when the elevated trace gas concentration was detected, the altitude when the elevated trace gas concentration was detected, the temperature when the elevated trace gas concentration was detected, the wind speed when the elevated trace gas concentration was detected, the wind direction when the elevated trace gas concentration was detected, and the pressure when the elevated trace gas concentration was detected from the unmanned vehicle.

12. The system of claim 1, wherein processing the first data packet and the second data packet includes: the timestamp for when the elevated trace gas concentration was detected, the sensor time when the elevated trace gas concentration was detected, and the trace gas concentration.

13. The system of claim 12, wherein processing the first data packet and the second data packet further includes: the latitude and longitude when the elevated trace gas concentration was detected and the altitude when the elevated trace gas concentration was detected.

14. The system of claim 13, wherein processing the first data packet and the second data packet further includes: the temperature when the elevated trace gas concentration was detected and the pressure when the elevated trace gas concentration was detected.

15. A method comprising:
detecting, by at least one trace gas sensor, an elevated trace gas concentration;
generating, by the at least one trace gas sensor, a first data and a second data packet of two or more data packets, wherein the first data packet and the second data packet comprise trace gas concentration data;
receiving, by a processor of a control electronics, the first data packet and the second data packet; and
processing, by the processor of the control electronics, the first data packet and the second data packet to include at least one of: a timestamp for when the elevated trace gas concentration was detected, a sensor time when the elevated trace gas concentration was detected, a trace gas concentration, a latitude and longitude when the elevated trace gas concentration was detected, an altitude when the elevated trace gas concentration was detected, a temperature when the elevated trace gas concentration was detected, a wind speed when the elevated trace gas concentration was detected, a wind direction when the elevated trace gas concentration was detected, and a pressure when the elevated trace gas concentration was detected.

16. The method of claim 15, further comprising:
providing power to the at least one trace gas sensor via the control electronics.

17. The method of claim 15, further comprising:
storing, by the processor of the control electronics, the first data packet in a memory of the control electronics; and
transmitting, by the processor of the control electronics, the second data packet to a ground control system (GCS) via a transmitter of the control electronics.

18. The method of claim 17, further comprising:
receiving, by a processor of the ground control system, the second data packet.

19. The method of claim 18, further comprising:
displaying, by the processor of the ground control system, at least one of: a map showing a location of the at least one trace gas sensor and a graph of the elevated trace gas concentration.
